(19) United States
(12) Patent Application Publication
(10) Pub. No.: US 2005/0059930 A1
(43) Pub. Date: Mar. 17, 2005

(54) Method and apparatus for localized drug delivery

(76) Inventors: Michi Garrison, Half Moon Bay, CA (US); Peter F. Campbell, Santa Clara, CA (US); Steve Salmon, Napa, CA (US)

Correspondence Address: ORRICK, HERRINGTON & SUTCLIFFE, LLP, 4 PARK PLAZA, SUITE 1600, IRVINE, CA, 92614-2558, US

(21) Appl. No.: 10/664,171

(22) Filed: Sep. 16, 2003

(51) Int. Cl. A61M 29/00

(52) U.S. Cl. 604/101.04

(57) ABSTRACT

A catheter system for localized administration of agents through the wall of a blood vessel is provided. Various catheter system constructions which use at least two expandable occluding elements to create the localized site are provided. The catheter system may include a catheter with a variable stiffness along its length. The catheter system may also include a hollow guide wire which is coupled to an expandable occluding element.

### FIELD OF THE INVENTION

The present invention relates generally to intravascular drug delivery to localized regions. The invention includes a catheter device having two or more occluding members, preferably balloons, associated therewith.

### BACKGROUND OF THE INVENTION

Methods for localized drug delivery are disclosed in Yock et al. U.S. Pat. No. 6,346,098, which is incorporated by reference, in its entirety, herein. The aforesaid Yock et al. patent describes several ways in which a pressurized system can be used to accomplish retrograde perfusion, alone or in conjunction with other modalities, e.g., energy, to cause disruption or increased porosity in a localized region of the wall of a blood vessel whereby an agent, e.g., a therapeutic substance, is caused to pass through the wall of a blood vessel to produce the desired effect in the tissue surrounding the localized delivery site. Angiogenesis and myogenesis are two particularly desirable uses of the Yock et al. method. Given the desirability of the effective use of that method, there remained a need for apparatus which would improve the effectiveness of the method and for improvements in the method itself. It is noted that Corday et al. U.S. Pat. Nos. 4,689,041 and 5,033,998 make use of a catheter having an occluding balloon at its distal end for retrograde venous injection of fluids into a blockaded region of the heart which has become inaccessible by reason of an occluded artery. The method of Corday et al. involves placing the balloon into the coronary sinus and directing fluid retrograde into all veins of the heart. Since the objective of Corday et al. is to deliver cardioplegic solution to the entire heart, the described system would appear to be suited for its purpose. However, it would not be useful to achieve the objectives of Yock et al. U.S. Pat. No. 6,346,098 which are centered on localized delivery through the wall of a blood vessel.

The patent to Glickman, U.S. Pat. No. 5,919,163, which is incorporated herein by reference, describes the use of a double balloon catheter to isolate a tumor for chemotherapy treatment.

### SUMMARY OF INVENTION

The apparatus of the present invention comprises a catheter system for delivery of an agent which catheter system has two occluding members, preferably balloons, which function to isolate an axial region within a blood vessel whereby the delivery of an agent through the blood vessel wall will take place only in the localized region. In this catheter system, at least two members, each of which may be a catheter, are used to carry each of the occluding members to the desired location in the blood vessel. At least one of the catheters will have a relatively stiff proximal region, a softer intermediate section and a still softer distal section. In a preferred embodiment, the catheter system comprises a telescoping assembly of two catheters, each provided with an occluding member whereby the length of the isolated region may be varied. In one embodiment thereof, a lumen is provided for infusion in the space between the two balloons and the catheter carrying the distal balloon moves inside the catheter carrying the proximal balloon.

In a further embodiment of the catheter system, the balloons are fabricated from a compliant material and have a variable diameter depending on inflation volume and/or pressure. Such materials include elastic polymers such as elastomeric polyurethane, silicone polymers, synthetic rubbers such as polyneoprene, neoprene and polybutylene, thermoplastic elastomers and other elastic materials well known to those skilled in the art.

In another embodiment, the system is provided with a pressure or other sensor which may be located in the catheter with the distal occlusion balloon. The sensor can be associated with an additional lumen which is provided for this purpose.

In still another embodiment, the system can be constructed such that the catheter with the proximal occlusion device is placed first using a guide wire and/or malleable stylet, such that this catheter acts as a guide for the catheter having the distal occlusion device. It is desirable for the catheter with the proximal occlusion device to be placed in the coronary sinus and certain physical characteristics are desirable for this purpose. These characteristics include a reinforced shaft which can transmit torque in its proximal region (e.g., approximately 50 cm) which does not enter the vasculature. The distal end is more flexible thereby enabling tracking into the venous anatomy. Additionally, the catheter shaft will have a built-in curve, so that the catheter is pointing the correct way to facilitate making a turn into the coronary sinus.

The present invention also includes a system in which the catheter with the distal occlusion device and the catheter with the proximal occlusion device are placed such that the catheter with the distal occlusion device is placed first and acts as a rail for the proximal catheter to advance.

The catheter system of the present invention may use a coaxial or dual lumen construction for the catheter having the proximal occlusion device and may use a tri-lumen construction for the catheter with the distal occlusion device. In another embodiment, the infusion lumen is the annular space between the two catheters. The amount of space will depend on the infusion flow rate desired. Radio opaque markers may be added to one or both catheters to mark desired points on catheters, e.g., the distal region of each catheter and/or the proximal position of the distal occlusion device. This will help catheter positioning and accurate measurement of the infusion space. Infusion pressure may be regulated passively, e.g., with a spring-controlled reservoir, or actively, e.g., using pressure from the catheter with the distal occlusion device to control a spring force or other mechanism for regulating infusion flow rate.

In still another embodiment of the present invention, a guide wire having a lumen in communication with an occluding device, such as a balloon, located at a distal region of the guide wire may be used to deploy the distal occlusion device with a catheter which slides over the guide wire providing the proximal occlusion device, such as a balloon. In such a device, the guide wire will typically pass through a lumen in the shaft of the catheter, which lumen may extend for the full length of the catheter or some part thereof.

### DETAILED DESCRIPTION OF THE DRAWINGS

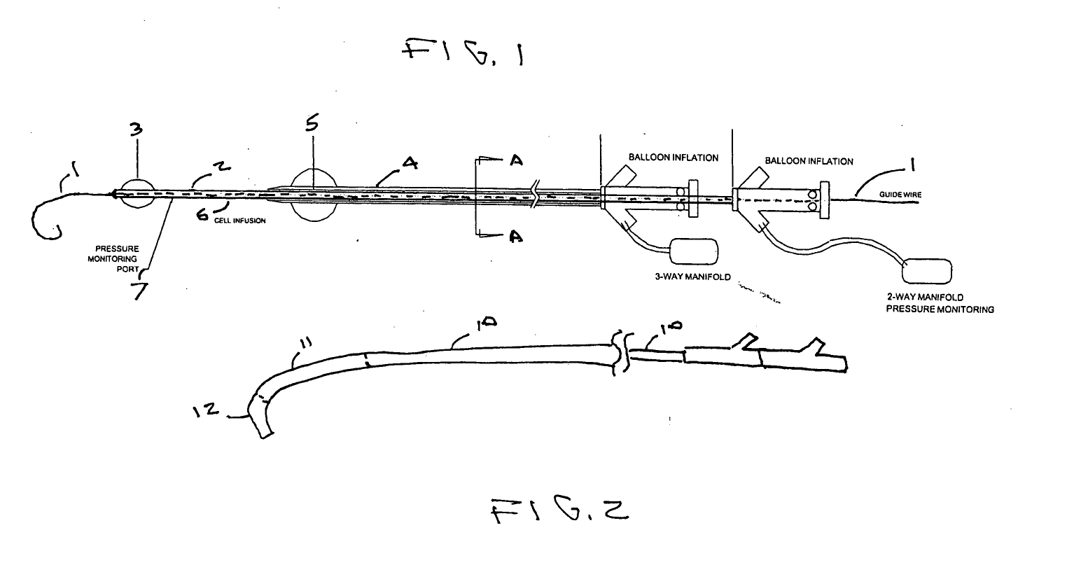

FIG. 1 is a schematic view of an embodiment of the catheter system of the present invention.

FIG. 2 shows the regions of the catheter which have different stiffnesses.

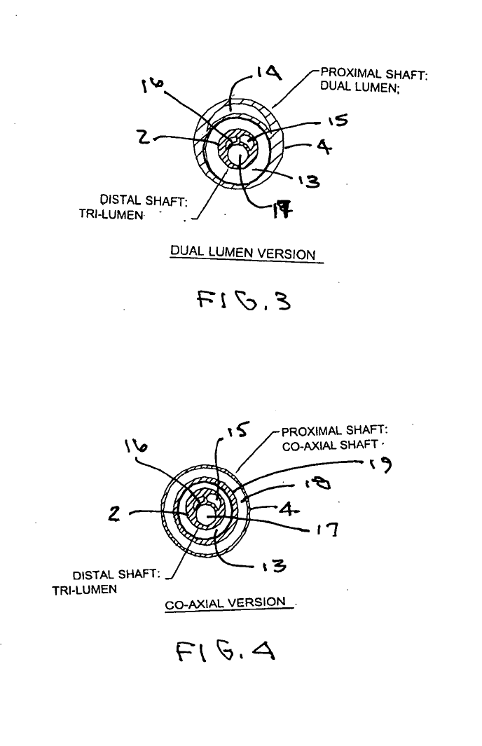

FIG. 3 illustrates a dual lumen version of the catheter system of the present invention.

FIG. 4 illustrates a coaxial version of the catheter system of the present invention.

### DETAILED DESCRIPTION OF THE INVENTION

As can be seen from FIG. 1, one embodiment of the present invention comprises two catheters, each of which is provided with a balloon. The catheter system is constructed such that it can pass over guide wire 1. Inner catheter 2 carries distal occlusion balloon 3. Similarly, outer catheter 4 carries occlusion balloon 5. The section 6 of inner catheter 2 is provided with infusion means, e.g., ports, through which a desired agent, e.g., cells, may be delivered to and administered to the patient through the blood vessel wall surrounding region 6 in the manner disclosed in Yock U.S. Pat. No. 6,346,098. As further shown in FIG. 1, the distal region of inner catheter 2 may also be provided with a pressure monitoring port which measures the pressure of the infusion medium.

FIG. 2 is a simplified illustration of inner catheter 2 of FIG. 1. Details of the catheter, such as the balloon, have been omitted for purposes of clarity. In FIG. 2, the relatively stiff proximal region of the catheter shaft is indicated by numeral 10. The softer intermediate section of the catheter is indicated by numeral 11 and the still softer distal region of the catheter is indicated by the numeral 12. The purpose of these three regions of different stiffness is to provide pushability and torque ability with the relatively stiff proximal region 10 and trackability with the softer intermediate and distal regions 11 and 12. This construction facilitates deployment of the distal region of the catheter in the coronary sinus of the patient which is desirable when the treatment of the patient will be for purposes of angiogenesis or myogenesis. In a preferred embodiment of the present invention, cells which will promote angiogenesis or myogenesis are delivered to a localized region of the heart.

In a preferred embodiment of the present invention, the inner catheter 2 is slidably associated with outer catheter 4 such that the space between balloon 3 and balloon 5 can be varied according to the circumstances of the desired treatment. Published United States patent application 2002/0188253, which is incorporated herein by reference, discloses a dual balloon system in which the catheters are slidable with relation to each other to thereby vary the space between the balloons as desired.

FIGS. 3 and 4 illustrate two different constructions of outer catheter 4 as well as other details of the device. In FIG. 3, the shaft of the outer catheter 4 is shown in a dual lumen configuration with main lumen 13 and balloon lumen 14. In this embodiment, the inflation medium for balloon 5 is passed through lumen 14. In this Figure, inner catheter 2 is also shown and has lumen 17 through which a guide wire or stylet may pass, as well as pressure monitoring lumen 15 and balloon inflation lumen 16.

In FIG. 4, catheter 4 is provided in a coaxial configuration such that it has main lumen 13 and annular space 18 which constitutes a passageway for the balloon inflation medium. Annular space 18 is formed by inner wall 19 which is spaced from the outer wall of catheter 4. In this embodiment, the structure of inner catheter 2 remains the same.

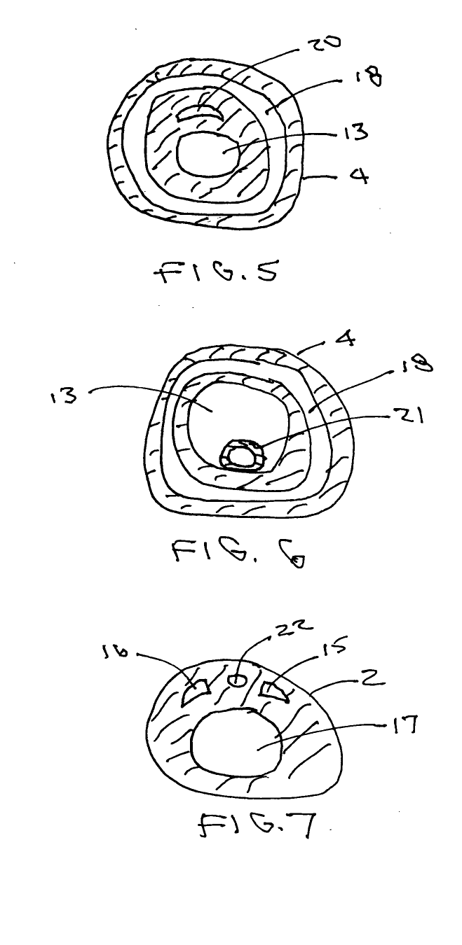

In the embodiments of FIGS. 4 and 5, the lumen 13 will be used both for tracking over a guide wire or previously-installed catheter and as a conduit for the infusion medium used to deploy the agent through the blood vessel wall at the desired location.

FIGS. 5 and 6 show alternate embodiments of outer catheter 4. As shown in FIG. 5, catheter 4 is provided with an additional lumen 20 which may be used for infusion or such as the purpose as may be desired. In FIG. 6, catheter 4 is provided with a small tube 21 which may be fabricated from any suitable metal or polymer material, e.g., stainless steel, nickel-titanium alloys, polyimides, and may serve as an additional infusion device or for such other purpose as may be desired.

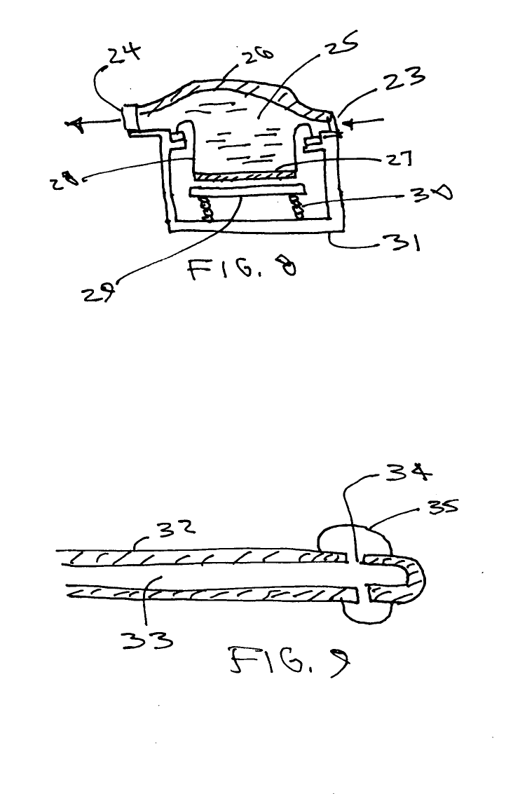

FIG. 7 illustrates, in cross section, a further embodiment of inner catheter 2 which is provided with an additional lumen 22 which may be used for infusion or such other purposes as may be desired.

All of the catheters shown in FIGS. 1-7 may be circular in cross section or may have other shapes such as elliptical or irregular.

The devices of the present invention may be provided with a pressure regulator to maintain a desired infusion pressure. Typically, an infusion pressure at the infusion site of 100-200 mmHg is desired, but greater or lesser pressures may be employed. The pressure regulator can usefully be attached to the catheter system between the infusion port on the catheter and a syringe or other means used to infuse the desired agent under pressure. The desired pressure at the regulator may be calculated from the desired pressure at the infusion site according to engineering principles well known to those skilled in the art. A pressure regulator useful with the catheter system of the present invention is illustrated in FIG. 8. The direction of fluid flow is indicated by the arrows shown adjacent the inlet 23 and the outlet 24 of the pressure regulator. The infusion fluid passes through cavity 25 in the pressure regulator which is formed by wall 26 and diaphragm 27 and flexible element 28. In a preferred embodiment, the diaphragm is circular in configuration.

Plate 29 is coupled to spring element 30 which may be a coil, leaf or other type of spring. A coil spring is illustrated. The spring is also coupled to the shell 31 of the pressure regulator. Pressure is regulated by the counter forces of the pressure of the fluid in cavity 25 and the pressure exerted by spring 30. When the pressure in cavity 25 exceeds the desired pressure, diaphragm 27 will be brought into contact with plate 29 and the spring force in spring 30 will counter undesired over pressurization in cavity 25.

FIG. 9 illustrates still another embodiment of the present invention in which a hollow guide wire is used instead of the inner catheter 2 shown in FIG. 1. In the embodiment of FIG. 9, the guide wire 32 is provided with lumen 33 and with one or more apertures 34 in its distal region. A balloon 35 is also coupled to the guide wire 32 such that inflation pressure for the balloon can be transmitted through lumen 33 and at least one aperture 34. In this embodiment, outer catheter 4 will be deployed over guide wire 32 and the balloon or other occluding device coupled to guide wire 32 will constitute the distal occluding device of the system.

1. A catheter system for creating an isolated blood vessel segment comprising:
a first catheter and a second catheter, each having a proximal and a distal end,
said first catheter having a first expandable occlusion device associated therewith,
said second catheter having a second expandable occlusion device associated therewith and being adapted to expand said second occlusion device distally of the first occlusion device on the first catheter,
said second catheter being slidably housed within a first lumen in said first catheter such the distance between said first and second occlusion device may be varied,
the occlusion devices being expandable to engage a wall of a blood vessel thereby substantially isolating an interior segment of a desired extent between said first and second occlusion devices, and at least one of said catheters having a relatively stiff proximal region, a softer intermediate region and a still softer distal region.

2. A catheter system for creating an isolated blood vessel segment comprising:
a catheter and a guide wire each having a proximal and a distal end,
said catheter having a first expandable occlusion device associated therewith,
said guide wire having a second expandable occlusion device associated therewith and being adapted to expand said second occlusion device distally of the first occlusion device on the first catheter,
said catheter being slidably mounted on said guide wire,
the occlusion devices being expandable to engage a wall of a blood vessel thereby substantially isolating an interior segment of a desired extend between said first and second occlusion devices.

3. The catheter system of claim 1 wherein the system is provided with a pressure regulator.

4. The catheter system of claim 1 wherein said second catheter is spaced from the inner wall of said first catheter to create a coaxial lumen surrounding a centrally located lumen.

5. The catheter system of claim 1 wherein said second catheter is provided with three lumens.

6. The catheter system of claim 4 wherein said centrally located lumen is provided with an axially extending tube.
